(19) United States
(12) Patent Application Publication
(10) Pub. No.: US 2015/0265355 A1
(43) Pub. Date: Sep. 24, 2015

(54) ENDOSCOPIC INSTRUMENT FOR THE CONNECTION TO AN OPERATION ROBOT

(71) Applicant: Richard Wolf GmbH, Knittlingen (DE)

(72) Inventors: Stephan PRESTEL, Rheinstetten (DE); Sören Münnig, Walzbachtal (DE); Elmar TEICHTMANN, Bretten (DE); Frank WEHRHEIM, Bretten (DE)

(21) Appl. No.: 14/660,369

(22) Filed: Mar. 17, 2015

(30) Foreign Application Priority Data
Mar. 18, 2014 (DE) 10 2014 205 036.7

(51) Int. Cl. A61B 19/00; A61B 17/00

(52) U.S. Cl. CPC A61B 19/2203; A61B 17/00234; A61B 2019/2215; A61B 2019/2234; A61B 2017/00473; A61B 2019/2242; A61B 2017/00477

(57) ABSTRACT

An endoscopic instrument for the connection to an operation robot. The endoscopic instrument includes an instrument housing, to which a shank with at least one instrument part arranged at the distal side of the shank connects distally. The instrument part and/or the shank are movable relative to the instrument housing and for this are each actively connected to control. The control can be coupled, via a translatorily movable coupling element coupled thereto and projecting out of the instrument housing, to a linear drive unit which can be connected to the instrument housing at the outer side.

### CROSS REFERENCE TO RELATED APPLICATIONS

This application claims the benefit of priority under 35 U.S.C. §119 of German Patent Application DE 10 2014 205 036.7 filed Mar. 18, 2014, the entire contents of which are incorporated herein by reference.

### FIELD OF THE INVENTION

The invention relates to an endoscopic instrument for the connection to an operation robot, with an instrument housing, with a shank which connects to the instrument housing, and with at least one instrument part arranged on a distal side of the shank, wherein the instrument part and/or the shank are movable relative to the instrument housing and for this are each actively connected to a control means (a control).

### BACKGROUND OF THE INVENTION

Operation robots are nowadays applied with an increasing number of surgical operations. Such an operation robot is known for example from US 2009/0234371 A1. This operation robot comprises several robot arms, at whose distal ends an endoscopic shank instrument is arranged each case, wherein this instrument is controlled by the operator from a console and serves for observing and/or manipulating on or in the body of a patient to be treated, depending on the type of instrument.

The instruments which are provided for manipulation comprise an instrument head which a tool arranged thereon, at their respective distal shank end. Known instruments are provided with an instrument head which can be bent relative to the shank, wherein the tool or a tool carrier with the tool and provided on the instrument head can also be bent (angled) with respect to the instrument head. Instruments which additionally comprise an instrument shank which can be rotated about their longitudinal axis are also widespread.

It is common to apply pull cables which are led through the shank into an instrument housing arranged at the proximal shank end, as control means, for the control of the rotation of the instrument shank and for the control of the bending of the instrument head as well as for the control of the tool or for actuating the instrument head and the tool. There, in the instrument housing, the pull cables are each non-positively coupled in movement to a rotatable actuation roller which is controlled by way of a rotatory drive motor. The actuation rollers are arranged next to one another in a common plane normal to their rotation axis. The drive motors which serve for actuating the pull cables are arranged in a drive unit which is releasably connectable to the instrument housing.

Typically, the number of actuation rollers which are arranged in the instrument housing and which is determined by the number of degrees of freedom of movement of the instrument head, has a significant influence on the size of the instrument housing. Moreover, various cable deflections which are necessary in the instrument housing moreover also have an additional disadvantageous influence on the dimensions of the instrument housing and increase the assembly effort on manufacture of the instrument. Thus, the instrument housing is already relatively large with an instrument, in whose instrument housing only four actuation rollers are arranged. This size of the instrument housing has been shown to be disadvantageous if several of these shank instruments are to be applied together in a restricted space, as is the case with single-port operations for example, with which the shank instruments are simultaneously led to the region of operation via a common body opening.

### SUMMARY OF THE INVENTION

Against this background, it is an object of the invention to provide an endoscopic instrument of the type described above, which has a constructionally simplified movement coupling of the control means for the control of at least one instrument part arranged distally of the shank, and/or of the shank, to a drive unit, and which permits the creation of a compact instrument housing.

The endoscopic instrument according to the invention is such an instrument which is used in combination with an operation robot. It comprises an instrument housing, onto which the shank connects at the distal side which is to say distally. At least one instrument part is arranged at the distal side of the shank usefully designed as a hollow shank. With regard to this instrument part, it can e.g. be the case of a tool or an instrument head serving as a tool carrier. The instrument part and/or the shank are movable relative to the instrument housing. In this context, one preferably envisages the instrument part being pivotable relative to the shank and to the instrument housing which is connected thereto, and the shank is rotatable about its longitudinal axis relative to the instrument housing. Several instrument parts can further preferably be arranged at the distal side of the shank and these are pivotable relative to one another and relative to the shank. Inasmuch as this is concerned, if hereinafter one speaks of the instrument part, this is also to be understood as several instrument parts.

The instrument part and/or the shank are actively connected to control means which are actuatable in the instrument housing, for the movement control. Usefully, a pull means which is led from the instrument part through the shank into the instrument housing is provided as control means, at least for the instrument part which is arranged at the distal side of the shank. The actuation of the control means for the instrument part and/or for the shank is effected via drive motors which are actively connected thereto and are arranged in a drive unit of the instrument which is connectable to the instrument housing, and subject the control means to a pulling force for movement control of the instrument, if with regard to the control means, it is the case of a pull means.

The basic idea of the invention lies in coupling the control means which are actively connected to the instrument part arranged distally of the shank and/or to the shank, in each case via a translatorily movable coupling element which is coupled in movement to these means, to a linear drive unit which can be connected onto the instrument housing at the outer side. The coupling element is preferably arranged in the instrument housing such that it projects out of the instrument housing.

In combination with the pull means which are preferably used for the control of the instrument part arranged at the distal side of the shank, this means that a translation of the coupling element which is produced by the linear drive unit can be transmitted directly onto the pull means given a suitable design. Accordingly, the actuation rollers which until now are to be arranged in the instrument housing are no longer necessary, which leads to a considerable gain in space in the instrument housing, which is to say the creation of a comparatively small instrument housing, wherein the number of the individual components which were necessary until now for the movement transmission of the drive motor onto the control means can be significantly reduced.

The instrument housing is advantageously designed in an essentially completely closed manner for the protection of the control means which are located therein, so that a part of the coupling element is to be led through an outer wall of the instrument housing. For this, usefully an opening corresponding to the movement path of the coupling element is formed on the outer wall of the instrument housing. The connection of the linear drive unit to the instrument housing and thus the coupling of the linear drive movement to the coupling element are usefully releasable in a repeated manner, in order to be able to separate the linear drive unit from the remaining instrument for cleaning or maintenance purposes for example.

The coupling element is preferably arranged on a track-guided pull slide (carriage) in the instrument housing. A linear guide, in which the pull slide is displaceably guided, is accordingly preferably provided in the instrument housing. E.g. hollow profile rails or two guide strips which are aligned parallel to one another can serve as track guidance for the pull slides in a manner which is simple with regard to the design. The track guide is usefully aligned in the movement direction of the pull means. The pull slide which is led in the track guide is directly connected to the control means, in a particularly space-saving manner.

The linear drive unit which can be coupled to the coupling element can be formed by a fluidically actuatable cylinder or by a linear motor which can be coupled to the coupling element in a direct manner via a coupling device which is connected thereto. A linear drive unit which comprises at least one rotatory drive motor, subsequent to which a gear designed for the conversion of the rotation movement of the motor shaft into a translation movement is arranged, is however preferably provided. An electric motor is preferably provided as a drive motor, but a fluidically actuated motor can also be used as the case may be.

With regard to the gear which is arranged subsequently to the drive motor, it is preferably the case of a rack-and-pinion gear, whose rack is coupled in movement to a coupling device which is positively connectable to the coupling element on the instrument housing side. In this case, a pinion meshing with the rack is usefully arranged on the motor shaft of the drive motor, on the drive side. The linear drive unit advantageously comprises an essentially closed housing, in which at least the rack-and-pinion gear arranged subsequently to the drive motor, and the coupling device coupled in movement thereto are arranged. Hereby, usefully at least one opening is formed on an outer wall of the housing which lies in the connection direction of this housing to the instrument housing, through which opening the positive connection of the coupling element arranged on the instrument housing side to the coupling device on the linear drive side can be effected.

The rack of the rack-and-pinion gear is preferably coupled in movement to a drive slide (drive carriage) which is track-guided in the linear drive unit and on which the coupling device is formed. The rack can preferably be connected to the drive slide in a direct manner or be formed directly on the drive slide. A guide track for the drive slide and which is arranged in the linear drive unit is usefully aligned corresponding to the movement path of the coupling element provided on the instrument housing side. Usefully, an opening is formed on an outer wall facing the instrument housing, on the preferably envisaged housing of the linear drive unit, through which opening the coupling device formed on the drive slide at least partly projects out of the housing, in order to permit a coupling of the coupling element to the coupling device.

The coupling element, in a constructionally simple manner, is a projection projecting out of the instrument housing, wherein a recess for the positive receiving of the projection is formed on the coupling device. The projection which is provided on the part of the instrument housing and the recess which is formed on the coupling device on the liner drive side are hereby usefully arranged in a manner such that the projection engages into the recess formed on the coupling device, on connection of the linear drive unit to the instrument housing, wherein a reference travel of the drive slide can be necessary for this as the case may be.

The coupling element is advantageously resiliently mounted on the slide in the connection direction of the instrument housing and the linear drive unit, in order to permit the engagement of the projection into the recess of the coupling device after a reference travel of the drive slide. This design permits the evasion of the projection when the projection arranged on the instrument housing side, and the recess formed on the coupling device arranged on the linear drive side are not arranged in a congruent manner on connection of the linear drive unit to the instrument housing, wherein the projection after a reference travel of the drive slide locks into the recess in a manner impinged by spring force. A further measure which is advantageous in this respect lies in the fact that the regions adjacent the recess forming guide ramps for the coupling element, on the drive slides, so that the reference travel of the drive slide is not inhibited by the projection.

In particular, if with regard to the instrument part arranged at the distal side, it is the case of an instrument part pivotable relative to the shank, preferably at least one pull cable is provided for this instrument part as a control means. Thus, two pull cables acting antagonistically upon the instrument part can be fastened on the instrument part in a direct or indirect manner, and these pull cables are led through the shank into the instrument housing, where they are coupled in movement in each case to a coupling element able to be coupled to a drive motor of the linear drive unit.

However, a design with which only one pull cable, whose two ends are connected to the instrument part in an antagonistically acting manner, is provided as a control means for the instrument part arranged at the distal side of the shank is preferred. In this case, only one coupling element coupled in movement to the pull cable and, on the part of the linear drive unit, only one drive motor are required for the movement control of the instrument part arranged at the distal side of the shank in this case. The pull cable is hereby advantageously guided in the instrument housing in a deflection roller guide, wherein the movement coupling of the pull cable to the coupling element is envisaged in a region between the shank and a deflection roller which is distanced furthest in the pull direction from the instrument part which is to be controlled.

The coupling of movement of the pull cable to the coupling element is advantageously effected via a pull slide. Thus, the pull cable is preferably connected to a pull slide on which the coupling element is formed and which can be displaced in the pull direction of this pull cable. The pull slide is usefully guided in a guide track which is arranged in a manner corresponding to the pull direction of the pull cable, in the instrument housing.

A section of the pull cable is preferably formed by a pull rod. In this context, one envisages both ends of the pull cable being fastened at both ends of the pull rod. The pull cable is preferably fastened on the pull slide on the section formed by the pull rod, wherein the connection of the pull cable to the pull slide is favorably not designed in a rigid manner. Instead, the pull rod is advantageously resiliently mounted on the pull slide on two projections which are distanced to one another in the pull direction of the pull cable, in order to protect the pull cable from an overload.

The shank for the control of its rotation movement is preferably coupled in movement to a pinion which in the instrument housing meshes with a rack coupled in movement to a coupling element, despite the fact that a pull cable which is coupled in movement to a coupling element in the described manner could be used as a control means for the control of the rotation movement of the shank.

The coupling element is advantageously designed on a pull slide which is connected to the rack. Hereby, the coupling element, with which it is preferably the case of a projection, is arranged at a side of the rack which is away from the toothing and projects out of the instrument housing in the already described manner.

As has been already noted, the instrument according to the invention can comprise several movable instrument parts on the distal side of its shank. In a preferred design, the instrument according to the invention as movable instrument parts comprises an instrument head pivotably arranged at the distal end of the shank, and a tool carrier pivotably arranged on the instrument head relative to the instrument head. Further preferably, with regard to the tool arranged on the tool carrier it is the case of a jaw tool, so that two jaw parts which are pivotable relative to one another are arranged on the tool carrier. Advantageously, the instrument head, the tool carrier and the two jaw parts of the tool are each coupled in movement to a pull cable as control means, wherein the individual pull cables in the already described manner can be coupled via a coupling element which is coupled in movement thereto and is led out of the instrument housing, in each case to a drive motor of a linear drive unit connectable to the instrument housing.

The invention is hereinafter explained in more detail by way of embodiment examples which are represented in the drawing. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings, shown in a schematically simplified manner, and in different scales in each case:

FIG. 1 is a perspective plan view showing an endoscopic instrument for the connection to an operation robot, without a linear drive unit;

FIG. 2 is a perspective bottom view showing the endoscopic instrument according to FIG. 1 without a linear drive unit;

FIG. 3 is a perspective bottom view showing a proximal end region of the instrument according to FIG. 1, without a linear drive unit;

FIG. 4 is a perspective bottom view showing a proximal end region according to FIG. 3, omitting a part of the instrument housing;

FIG. 5 is a view according to FIG. 4, with a linear drive unit connected on the instrument housing;

FIG. 6 is a perspective plan view showing the linear drive unit according to FIG. 5;

FIG. 7 is a perspective representation showing a coupling element which is provided on the instrument side, in the condition coupled to a drive motor of the linear drive unit;

FIG. 8 is a perspective individual representation showing the coupling element according to FIG. 7;

FIG. 9 is a perspective representation showing a further embodiment of the coupling element;

FIG. 10 is a perspective representation, in a first view, showing the coupling element which is shown in FIG. 8, with an arrangement on a pull slide;

FIG. 11 is a perspective representation, in a second view, showing the coupling element represented in FIG. 8, with the arrangement on a pull slide;

FIG. 12 is a perspective representation, in a third view, showing the coupling element which is shown in FIG. 8, with an arrangement on a pull slide;

FIG. 13 is a perspective representation showing a drive train of the linear drive unit according to FIG. 6;

FIG. 14 is a lateral view showing the endoscopic instrument according to one of the preceding figures;

FIG. 15 is a perspective bottom view showing the instrument according to FIG. 14; and

FIG. 16 is a detail view from FIG. 4.

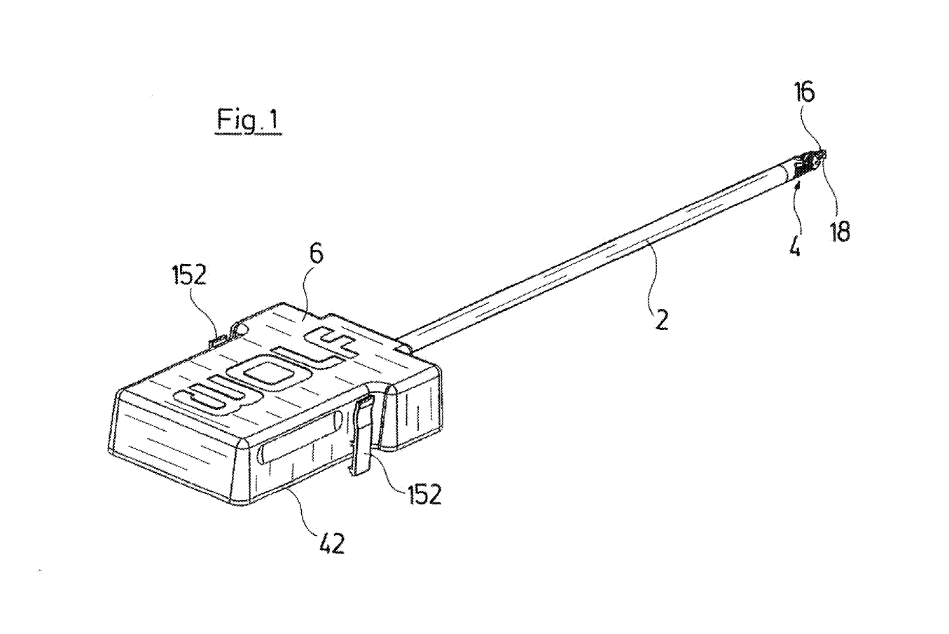

### DESCRIPTION OF THE PREFERRED EMBODIMENTS

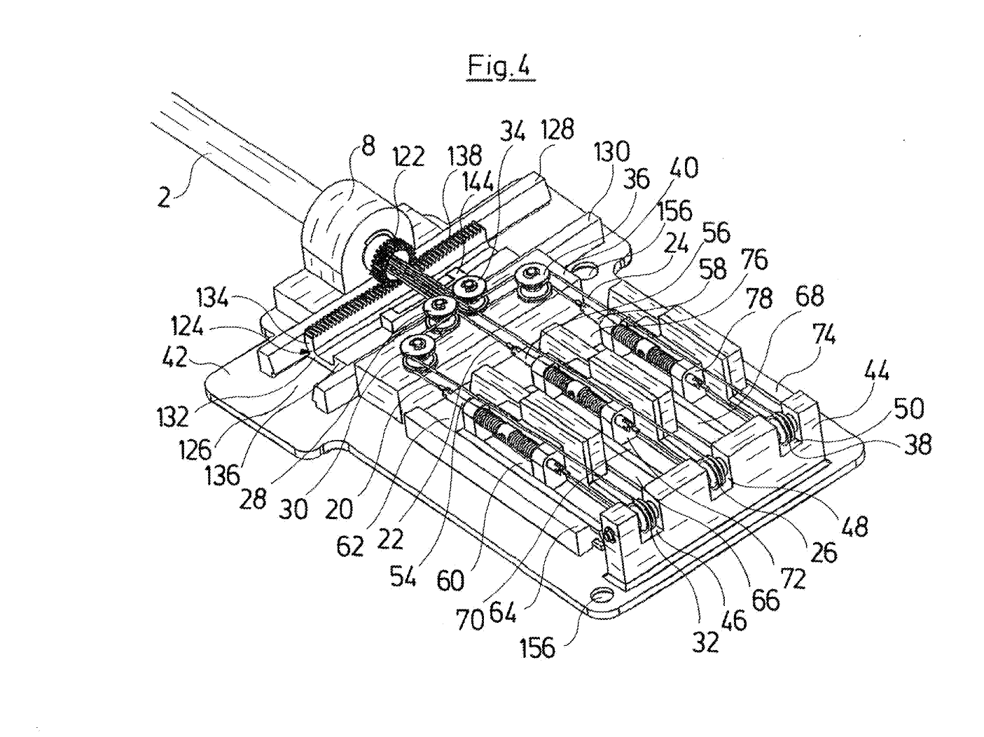

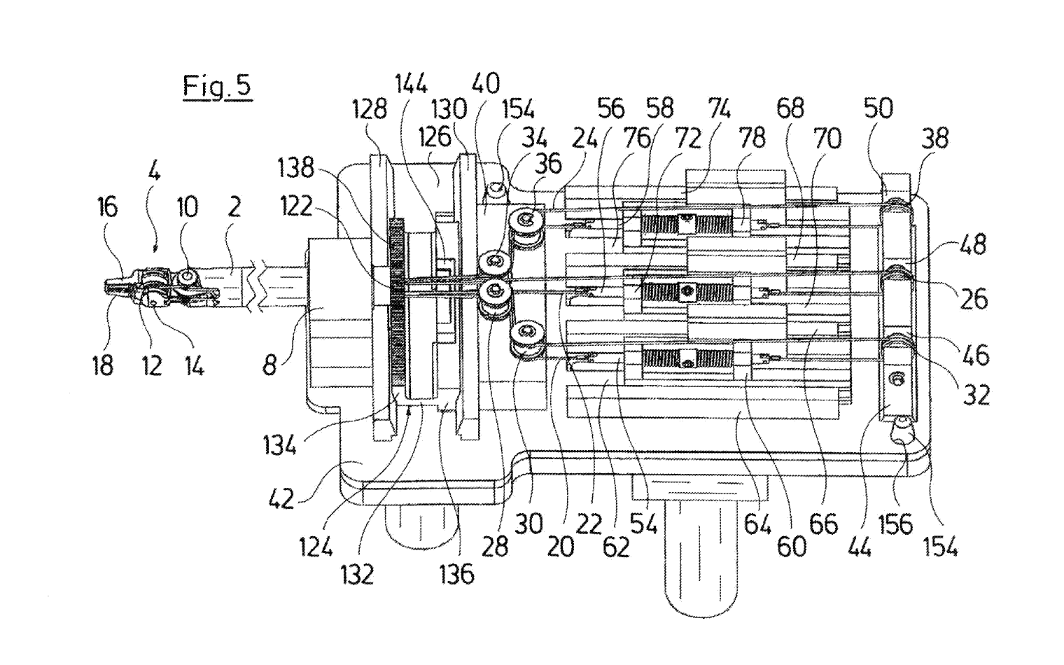

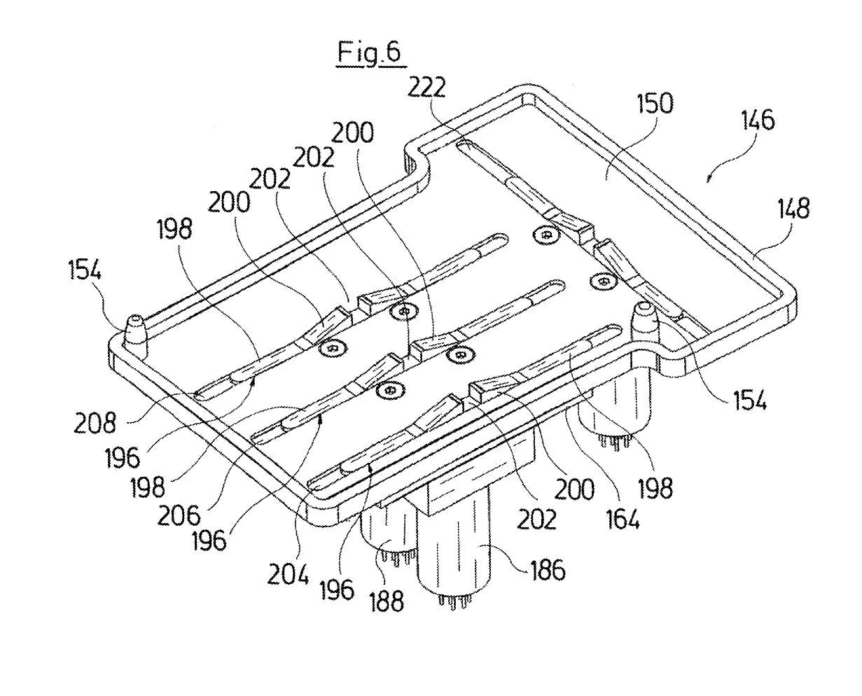

With regard to the endoscopic instrument represented in the drawing, it is the case of a shank instrument with a shank 2 which is designed as a hollow shank and on which an instrument head 4 is arranged at the distal side. An instrument housing 6, into which a proximal end region of the shank 2 engages and there in a mounting device 8 (FIGS. 4 and 5) is rotatably mounted relative to the instrument housing 6, is provided at the proximal side of the shank 2.

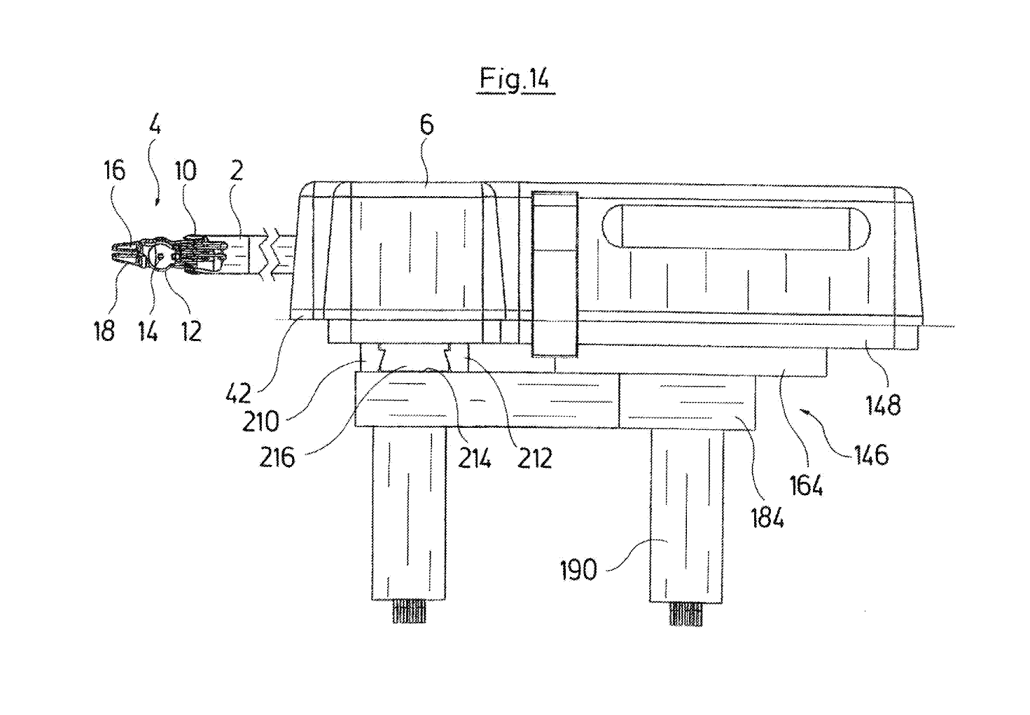

It is evident from FIGS. 5, 14 and 15 that the instrument head 6 is pivotably articulated on the shank 2 via a joint pin 10 and is pivotable in a plane transverse to the longitudinal extension of the shank 2. A tool carrier 12 is articulated on the instrument head 4 at the distal side via a joint pin 14, in a manner such that the tool carrier 12 can pivot in a plane normal to the pivot plane of the instrument head 4. The tool carrier 12 carries a jaw tool with two jaw parts 16 and 18 which are pivotable relative to one another.

The movement control of the instrument head 4, of the tool carrier 12 which is articulated thereon, and of the jaw parts 16 and 18 is effected via three pull cables 20, 22 and 24, wherein the instrument head 4, the tool carrier 12 and the two jaw parts 16 and 18 are each coupled in movement to one of the pull cables 20, 22 and 24 in an antagonistically acting manner, which is not evident from the drawing.

The pull cables 20, 22 and 24 are led through the shank 2 into the instrument housing 6 and there are each tensioned by way of a deflection roller arrangement. Hereby, the pull cable 24 merely wraps one deflection roller 26 which is arranged in the proximal end region of the instrument housing 6. The pull cable 22 on a deflection roller pair 28 which is arranged in the instrument housing 6 proximally of the proximal shank end, and on a deflection roller pair 30 arranged laterally distanced thereto, leads to a deflection roller 32 which is arranged next to the deflection roller 26 and around which it wraps. In a similar manner, the pull cable 24 is guided on a deflection roller pair 34 arranged in the instrument housing 6 proximally of the proximal shank end, and on a deflection roller pair 36 arranged laterally distanced thereto, to a deflection roller 38 which is arranged next to the deflection roller 26 and around which it wraps.

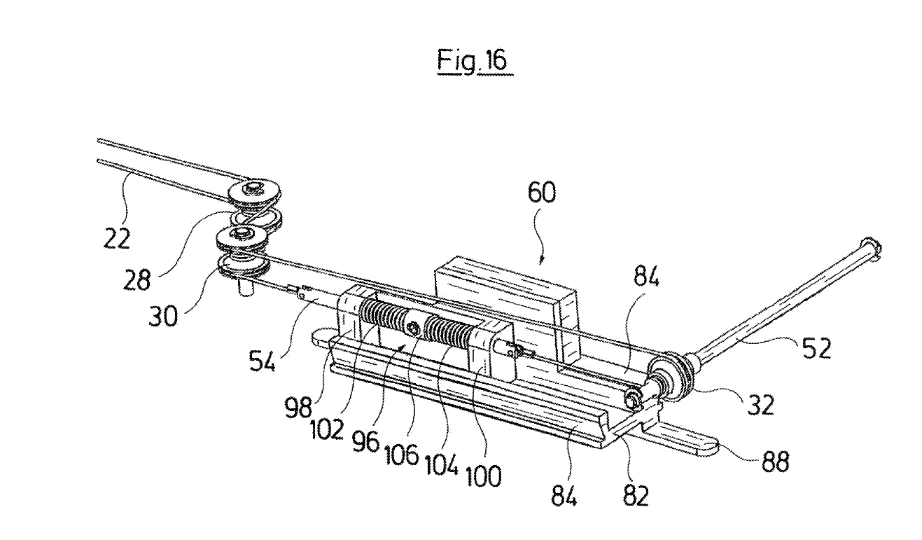

The deflection roller pairs 28, 30, 34 and 36 are mounted together on a mounting body 40 which is arranged on a base plate 42 of the instrument housing 6. The base plate 42 forms a part of the outer wall of the instrument housing 6. A mounting body 44 which is likewise arranged on the base plate 42 and at its side which is away from the base plate 42 is provided with three recesses 46, 48 and 50, in which the defection rollers 26, 32 and 38 are rotatably mounted on a common mounting pivot 52 (FIG. 16) serves for mounting the deflection rollers 26, 32 and 38.

The pull cable 20 in the region between the defection roller pair 30 and the defection roller 32 is guided in the instrument housing 6 parallel to the longitudinal extension of the shank 2. The pull cable 24 is likewise led parallel to the longitudinal extension of the shank 2, in the region between the deflection roller pair 36 and the defection roller 38. This also applies to the pull cable 22 which is guided in the instrument housing between the pull cables 20 and 24. The pull cables 20, 22 and 24 are each subjected to a pull force in this region, for the control of the instrument head 4, the tool carrier 12 as well as the jaw parts 16 and 18, and this will be dealt with in more detail hereinafter.

A section of the pull cable 20 is formed by a pull rod 52, in the region between the deflection roller pair 30 and the deflection roller 32. In a similar manner, a section of the pull cable 22 is also formed by a pull rod 56, and a section of the pull cable 24 in the region between the deflection roller pair 36 and the deflection roller 38 is formed by a pull rod 58.

The pull cable 20 in the region of the pull rod 54 is fastened on a pull slide 60, which in the instrument housing 6 is guided in a guide track 62 aligned parallel to the guiding of the pull cable 20 and formed by two guide strips 64 and 66 which are arranged on the base plate 42 of the instrument housing 6. The guide strips 64 and 66 as well as the pull slide 60 are designed for forming a swallowtail guide.

A guide strip 68 is arranged on the base plate 42 of the instrument housing 6, on the side of the guide strip 66 which is away from the guide strip 64, in a manner distanced to the guide strip 66. The guide strip 68 together with the guide strip 66 forms a guide track 70 which is designed as a swallowtail guide, in which a pull slide 72, on which the pull cable 22 is fastened via the pull rod 56 is linearly guided.

A further guide strip 74 is arranged on the base plate 42 at the side of the guide strip 68 which is away from the guide strip 66, in a manner distanced to this guide strip 68. The guide strip 74 together with the guide strip 68 forms a guide track 76 in the form of a swallowtail guide for a pull slide 78. The pull cable 24 is fastened on the pull slide 78 via the pull rod 58.

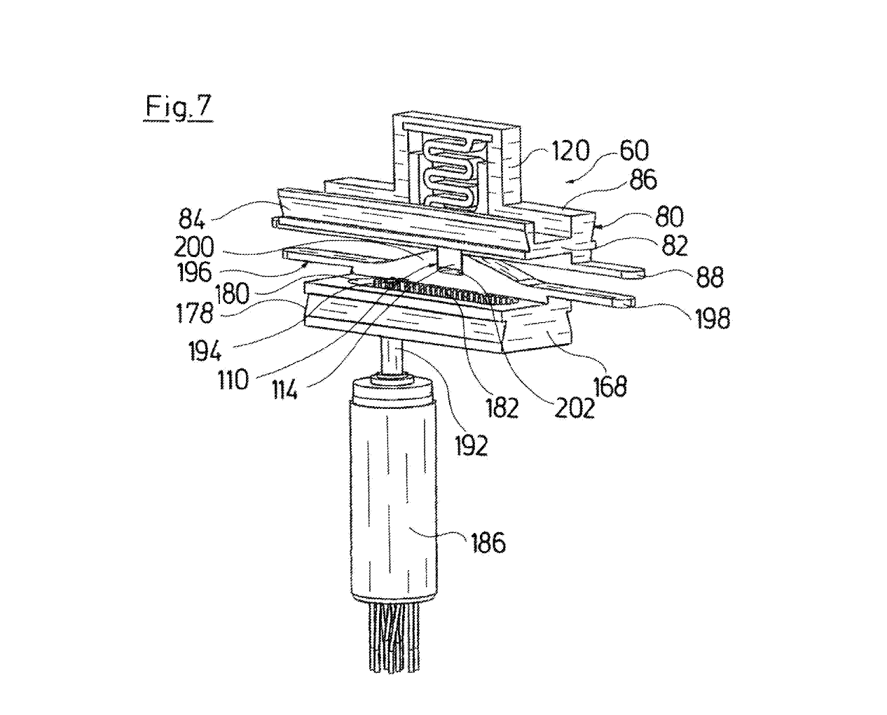

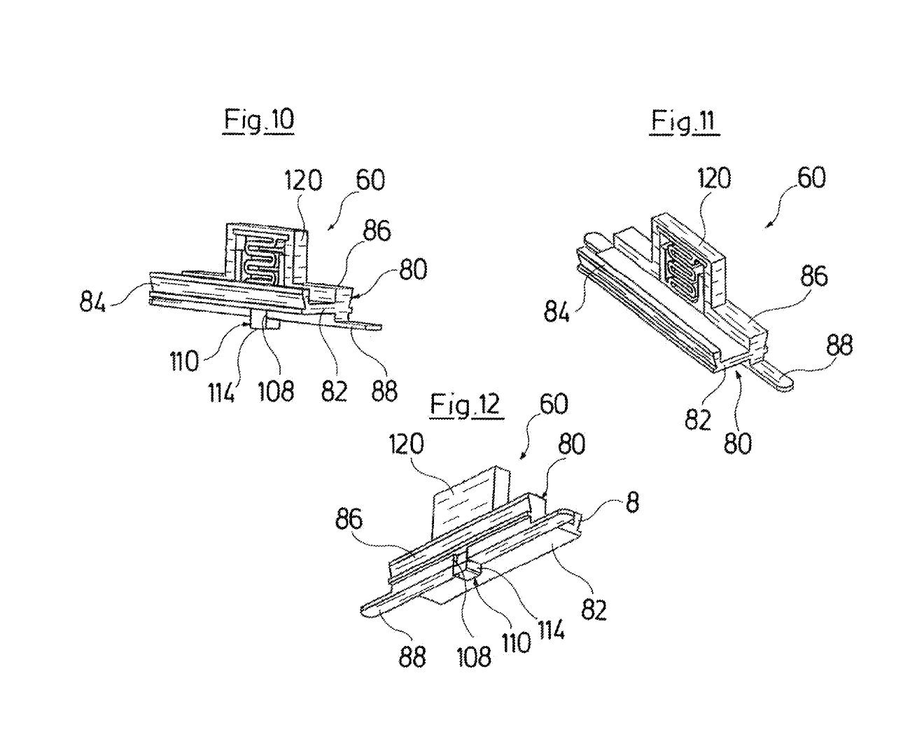

The construction of the pull slides 60, 72 and 78 is evident from the FIGS. 7, 10-12 as well as 16, in which the pull slide 60 is represented by way of example. The pull slides 72 and 78 are constructionally identical to the pull slide 60.

The pull slides 60, 72 and 78 each comprise a hollow-rail-like base body 80 with an essentially U-shaped cross section. The pull slides 60, 72 and 78 each lie on the base plate 42 of the instrument housing 6, via a flat base 82 formed on the base body 80. Walls 84 and 86 extend normally to the flat sides of the base 82, on the two longitudinal sides of the base 82. The outer sides of the walls 84 and 86 which are away from one another are each provided with bevellings for forming the swallowtail guide. The wall 86 extends beyond a flat side of the base 82 which faces the base plate 42 of the instrument housing 6. An elongate rail 88 is formed there on the wall 86, and this rail extends beyond the wall 86 in the longitudinal direction of this wall 86 at its two ends.

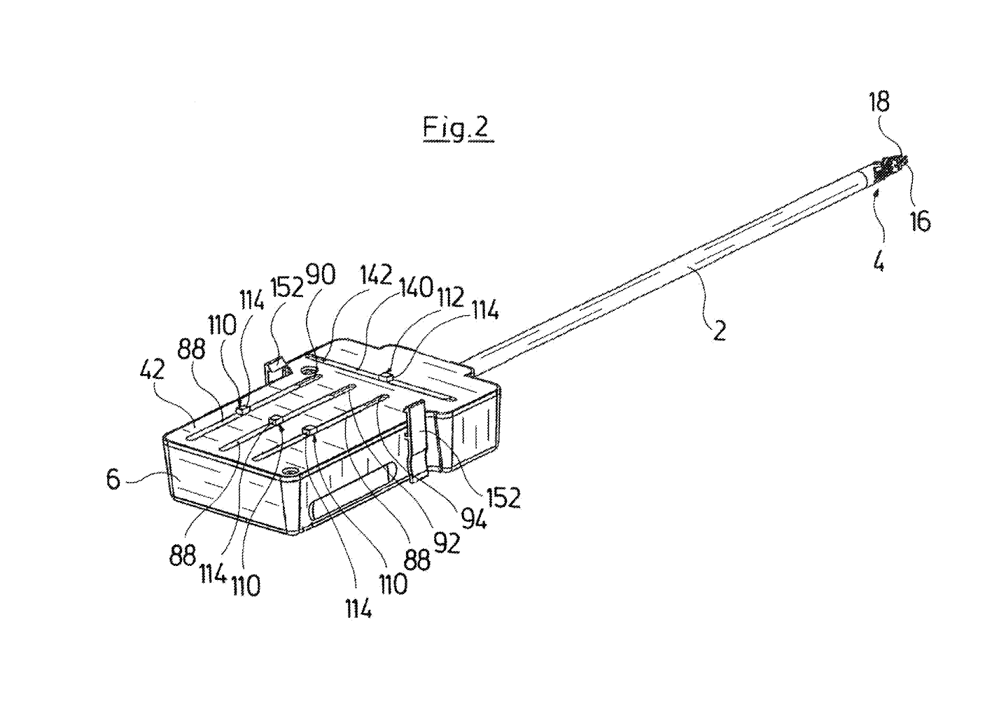

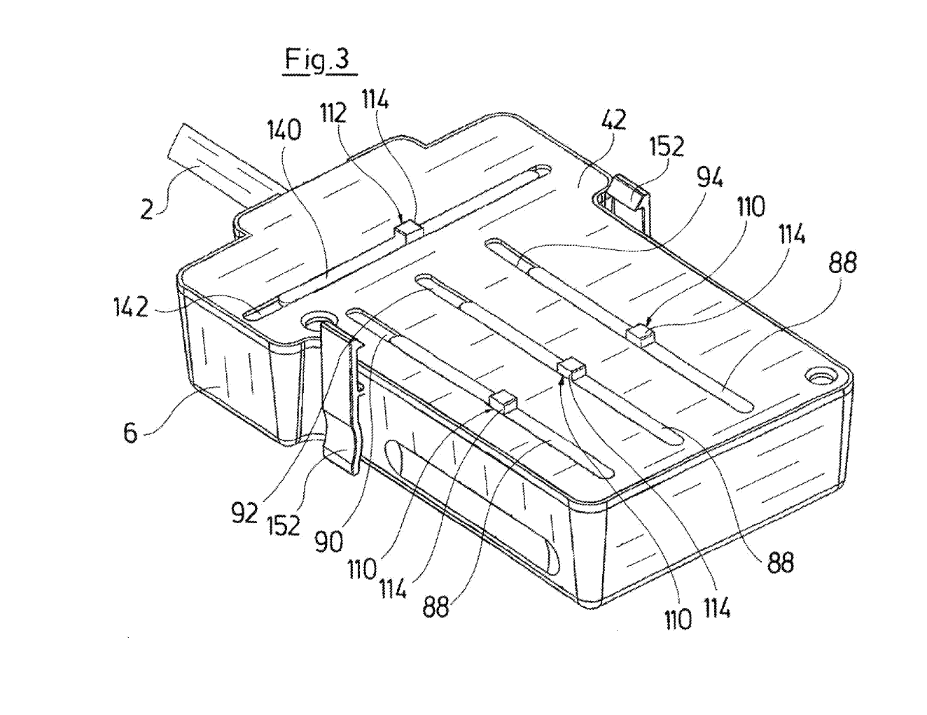

As is particularly evident from FIGS. 2 and 3, the rail 88 of the pull slide 60 engages into an elongate opening 90 which is formed on the base pate 42 of the instrument housing 6 and which is formed on this base plate 42 in the region of the guide track 62. In a manner corresponding to this, the rails 88 of the pull slide 72 engage into an opening 92 formed in the region of the guide track 70, and the rail 88 of the pull slide 78 engages into an opening 94 formed in the region of the guide track 76. The openings 90, 92 and 94 are in each case designed longer than the rails 88 of the pull slides 60, 72 and 78, in order to permit a linear displacement of the pull slides 60, 72 and 78.

In each case a fastening block 96 for fastening the pull rods 54, 56 and 58 of the pull cables 20, 22 and 24 is arranged on the flat side of the bases 82 of the pull slides 60, 72 and 88, said flat side being away from the base plate 42 of the instrument housing 6 (see in particular FIG. 16). The fastening blocks 96 each comprise two projections 98 and 100 which are distanced to one another in the longitudinal direction of the pull sides 60, 72 and 78 respectively and which project normally to the flat sides of the base 82. The two projections 98 and 100 are each provided with a bore which extends through the projections 98 and 100 in the longitudinal direction of the fastening block 96. The pull rods 54, 56 and 58 on the respective pull slides 60, 72 and 78 are led through the bores of the projections 98 and 100, wherein the pull rods 54, 56 and 58 in the intermediate space between the projections 98 and 100 are surrounded by two helical springs 102 and 104 which are arranged next to one another, as well as a fixation sleeve 106 arranged between the helical springs 102 and 104. The pull rods 54, 56 and 58 are fastened on the fastening block 96 of the respective slide 60, 72 and 78 via this arrangement of the helical springs 102 and 104 as well as of the fixation sleeve 106, wherein the helical springs 102 and 104 form an overload protection for the pull cables 20, 22 and 24.

It is evident from FIG. 12 that an opening 108 extending transversely to the longitudinal extension of the wall 86 through this wall is formed on the wall 86 of the pull slides 60, 72 and 78. A coupling element 110 which can be coupled to a drive motor of a linear drive unit 146 connectable to the instrument housing 6 is led through the opening 108, wherein this is yet explained in more detail hereinafter.

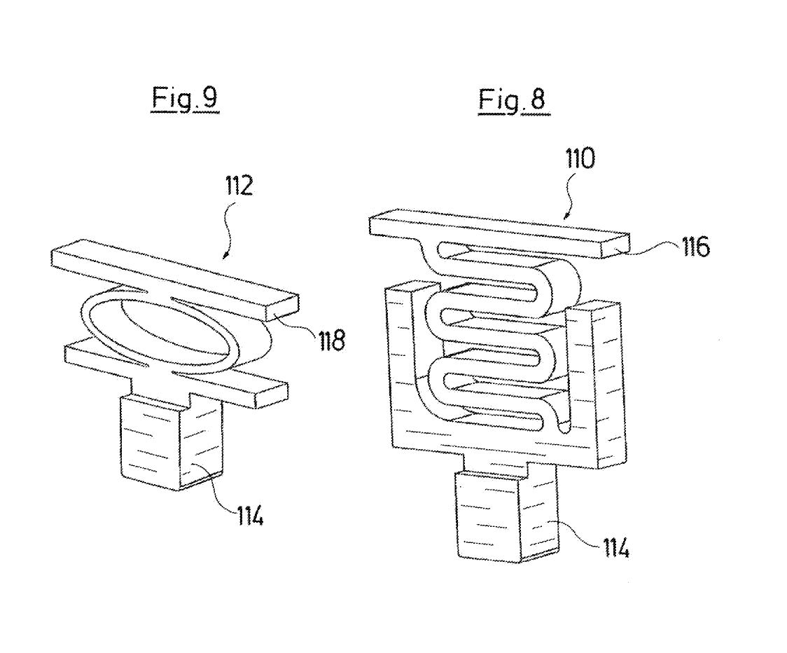

The coupling element 110 is represented in FIG. 8 and a further coupling element 112 is represented FIG. 9. Both coupling elements 110 and 112 have a positive-fit body in the form of a projection 114, on which a spring element 116 connects with regard to the coupling element 110, and a spring element 118 connects with regard to the coupling element 112. The spring element 116 of the coupling element 110 has a spring body which is shaped in a meandering manner, whereas the spring element 118 of the coupling element 112 has an annular spring body.

The arrangement of the coupling elements 110 on the pull slides 60, 72 and 78 is such that the positive fit body 114 of the coupling element 110 is led through the opening 108 formed on the wall 86 and projects at the outer side of the base plate 42 of the instrument housing 6 which is away from the pull slides 60, 72 and 78 respectively, whereas the spring element 116 of the coupling element 110 is supported in a frame 120 formed on the wall 86.

The shank 2 at its proximal end which projects into the instrument housing 6 proximally of the mounting device 8 is surrounded by a toothed ring 112 (FIGS. 4 and 5). The toothed ring 122 is connected to the shank 2 in a rotationally fixed manner. A pull slide 124 is displaceably arranged transversely to the longitudinal extension of the shank 2, between the toothed ring 122 and the base plate 42 of the instrument housing 6. Hereby, the pull slide 124 is guided in a guide track 126 which is formed by guide strips 128 and 130. The pull slide 124 comprises a base 132, via which it lies on the base plate 42 of the instrument housing 6. Walls 134 and 136 extend normally to the flat sides of the base 132, on the two longitudinal sides of the base 132. The outer sides of the walls 134 and 136 which are away from one another are each provided with bevellings for forming a swallowtail guide. The inner sides of the guide strips 128 and 130 which face one another comprise corresponding bevellings, in a manner corresponding to this. A rack 138 which is meshed by a toothed ring 122 is formed on an upper side of the wall 134 which away from the base 132.

The wall 136 of the pull slide 124 extends beyond a flat side of the base 132 which faces the base plate 42 of the instrument housing 6. An elongate rail 140 which extends beyond the wall 136 in the longitudinal direction of the wall 136 at its two ends is formed on the wall 136 there.

The rail 140 of the pull slide 124 engages into an elongate opening 142 which is formed on the base plate 42 of the instrument housing 6 and which is formed on the base plate 42 in the region of the guide track 126 (FIGS. 2 and 3). The opening 142 is aligned normally to the openings 90, 92 and 94. The opening 142 is designed longer than the rail 140 of the pull slide 124, in order to permit a linear displacement of the pull slide 124.

An opening extending transversely to the longitudinal extension of the wall 136 and through this is formed on the wall 136 of the pull slide 124, as with the pull slides 60, 72 and 78, but is not evident from the drawing. The coupling element 112 which is represented in FIG. 9 and which can likewise be coupled to a drive motor of a linear drive unit connectable to the instrument housing 6 is led through this opening.

The arrangement of the coupling element 112 on the pull slide 124 is such that the positive-fit body 114 of the coupling element 112 is led through the opening formed on the wall 136, and projects at the outer side of the base plate 42 of the instrument housing 6, said outer side being away from the pull slide 124, whereas the spring element 118 of the coupling element 112 is supported in a frame 144 formed on the wall 136.

As has already been noted, a linear drive unit 146 can be connected onto the instrument housing 6. The connection of this linear drive unit 146 onto the instrument housing 6 is effected via a housing part 148 of the linear drive unit 146 which can be connected to the instrument housing 6 by way of a clip connection. The housing part 148 is designed in an open manner at its side which faces the instrument housing 6.

The housing part 148 at the side which is away from the instrument housing 6 is closed by a base plate 150, whose dimensions correspond to the dimensions of the base plate 42 of the instrument housing 6. Two fastening clips 152 which positively and peripherally engage the housing part 148 connected to the instrument housing 6 are arranged on the instrument housing 6 on two side walls which are away from one another, for the releasably connection of the housing part 148 to the instrument housing 6. Two projections 154 which engage into holes 156 formed on the base plate 42 of the instrument housing 6 and which project in the direction of the instrument housing 6 are formed on the base plate 150 of the housing part 148, for simplifying the assembly of the instrument housing 6 and the housing part 148.

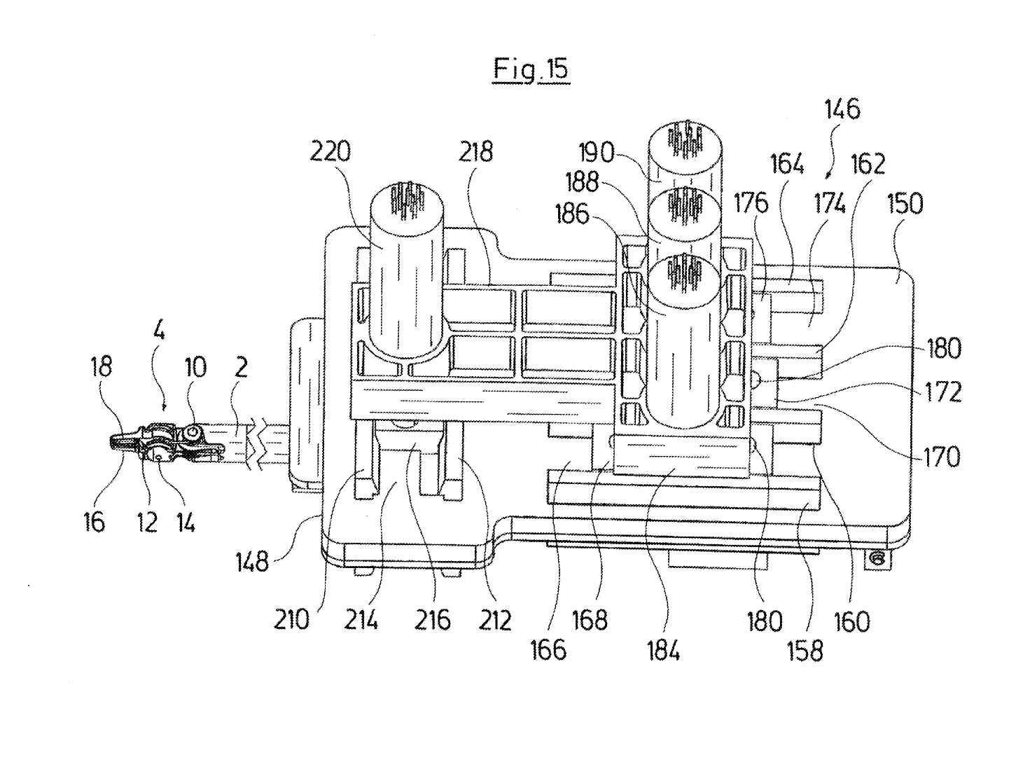

It is particularly evident from FIG. 15 that four guide rails 158, 160, 162 and 164 which are aligned parallel to the longitudinal extension of the shaft 2 and parallel to one another are arranged on the flat side of the base plate 150 of the housing part 148 of the linear drive unit 146, said flat side being away from the instrument housing 6. The guide rails 158 and 160 form a guide track 166, in which a drive slide 168 is linearly displaceably guided. The guide rails 160 and 162 form a guide track 170, in which a drive slide 172 is linearly displaceably guided. A guide track 174 for a drive slide 176 is formed by the guide rails 162 and 164.

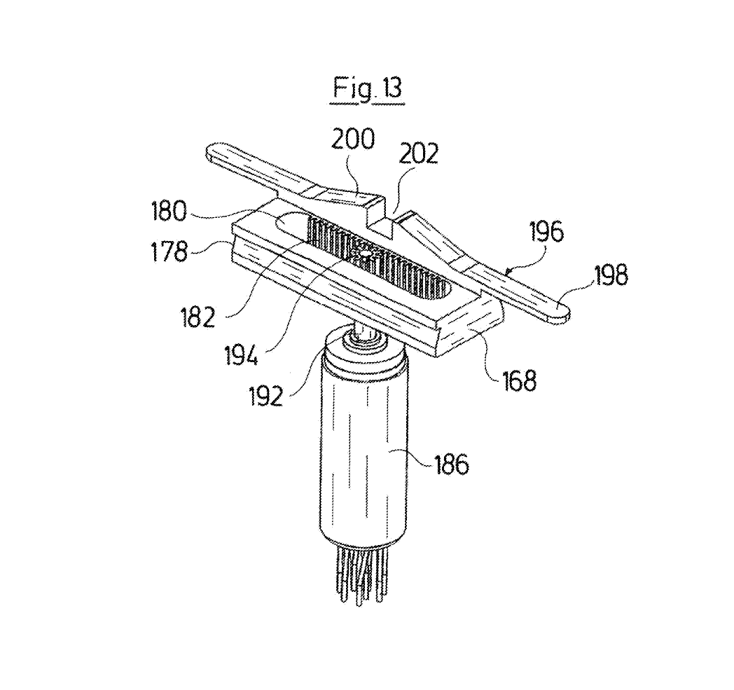

The drive slides 168, 172 and 176 are designed in a constructionally identical manner. Their design is evident from FIGS. 7 and 13, in which the drive slide 168 is shown in an exemplary manner. The drive slides 168, 172 and 176 are designed in an essentially cuboid manner, wherein at their longitudinal sides which are away from one another they comprise a wedge-like recess 178 extending over the entire length of the drive slide 168, 172 or 176. The recesses 178 in combination with a corresponding profiling on the inner sides of the guide rails 158, 160, 162 and 164 which face one another, serve for forming a swallowtail guide. An elongate hole 180 aligned in the longitudinal direction of the drive slide 168, 172 and 176 is moreover formed on the drive slides 168, 172 and 176 respectively. A longitudinal side of the elongate hole 180 is provided with a toothing and forms a rack 182.

A bearing block 184 which carries three electrically operated, rotatory drive motors 186, 188 as well as 190 is supported on the sides of the guide rails 158, 160, 162 and 164 which are away from the base plate 150 of the housing part 148. The arrangement of the drive motors 186, 188 and 190 on the bearing block 184 is such that a motor shaft 192 of the drive motor 186 engages into the elongate hole 180 formed on the drive slides 168, a motor shaft 192 of the drive motor 188 engages into the elongate hole 180 formed on the drive slide 172 and a motor shaft 192 of the drive motor 190 engages into the elongate hole 180 formed on the drive slide 176. A pinion 194 which meshes with the rack 182 in the elongate hole 180 of the related drive slide 168, 172 and 176 is arranged in each case on the ends of the motor shafts 192 of the drive motors 186, 188 and 190.

A coupling device 196 is arranged next to the elongate hole 180, on the flat side of the drive slides 168, 172 and 176 which is away from the drive motors 186, 188 and 190. The coupling device 196 is formed by an elongate rail 198 which extends at both longitudinal ends of the drive slides 168, 172 and 176 beyond this. A prominence 200 with two side surfaces tapering to one another at a shallow angle is formed in the region of the middle of the rail 198 at the side which is away from the drive slides 168, 172 and 176. A recess 202 extending in the direction of the drive slides 168, 172 and 176 is formed in the region of the middle of the prominence 200.

The coupling devices 196 of the drive slides 168, 172 and 176 engage into three elongate openings 204, 206 and 208 which are formed on the base plate 150 of the housing part 148. Hereby, the prominences 200 with the recess formed therein in the housing part 148 project in a freely accessible manner in the direction of the instrument housing 6.

Apart from the four guide rails 158, 160 162 a well as 164, two further guide rails 210 and 212 aligned normally to the guide rails 158, 160, 162 and 164 and parallel to one another are arranged on the flat side of the base plate 150 of the housing part 148 of the linear drive unit 146, said flat side facing away from the instrument housing 6. The guide rails 210 and 212 form a guide track 214, in which a drive slide 216 is linearly displaceably guided.

The drive slide 216 is designed in a constructionally identical manner to the drive slides 168, 172. A bearing block 218 carrying an electrically operated rotatory drive motor 220 is supported on the sides of the guide rails 210 and 212 which are away from the base pate 150 of the housing part 148. The bearing block 218 together with the bearing block 184 forms a common construction unit.

The arrangement of the drive motor 220 on the bearing block 218 is such that a motor shaft of the drive motor 220 engages into the elongate hole 180 formed on the drive slide 216, which is not evident from the drawing, wherein a pinion arranged at the end of the drive shaft meshes with the rack 182 in the elongate hole 180.

Next to the elongate hole 180, a coupling device 196 with an elongate rail 198 and with prominence 200 having a recess 202 and arranged on said rail is arranged on the flat side of the drive slide 216 which faces away from the drive motor 220. This coupling device 196 of the drive slide 216 engages into an elongate opening 222 which is formed on the base plate 150 of the housing part 148 and which is aligned normally to the openings 204, 206 and 208, wherein the prominence 200 with the recess 202 formed thereon projects in the housing part 148 in the direction of the instrument housing 6 in a freely accessible manner.

While specific embodiments of the invention have been shown and described in detail to illustrate the application of the principles of the invention, it will be understood that the invention may be embodied otherwise without departing from such principles.

### APPENDIX

List of reference numerals

| | |
|---|---|
| 2 | shank |
| 4 | instrument head |
| 6 | instrument housing |
| 8 | bearing device |
| 10 | joint pin |
| 12 | tool carrier |
| 14 | joint pin |
| 16 | jaw part |
| 18 | jaw part |
| 20 | pull cable |
| 22 | pull cable |
| 24 | pull cable |
| 26 | deflection roller |
| 28 | deflection roller pair |
| 30 | deflection roller pair |
| 32 | deflection roller |
| 34 | deflection roller pair |
| 36 | deflection roller pair |
| 38 | deflection roller |
| 40 | mounting body |
| 42 | base plate |
| 44 | mounting body |
| 46 | recess |
| 48 | recess |
| 50 | recess |
| 52 | bearing pivot |
| 54 | pull rod |
| 56 | pull rod |
| 58 | pull rod |
| 60 | pull slide |
| 62 | guide track |
| 64 | guide strip |
| 66 | guide strip |
| 68 | guide strip |
| 70 | guide track |
| 72 | pull slide |
| 74 | guide strip |
| 76 | guide track |
| 78 | pull slide |
| 80 | base body |
| 82 | base |
| 84 | wall |
| 86 | wall |
| 88 | rail |
| 90 | opening |
| 92 | opening |
| 94 | opening |
| 96 | fastening block |
| 98 | projection |
| 100 | projection |
| 102 | helical spring |
| 104 | helical spring |
| 106 | fixation screw |
| 108 | opening |
| 120 | coupling element |
| 112 | coupling element |
| 114 | projection |
| 116 | spring element |
| 118 | spring element |
| 120 | frame |
| 122 | toothed ring |
| 124 | pull slide |
| 126 | guide track |
| 128 | guide strip |
| 130 | guide strip |
| 132 | base |
| 134 | wall |
| 136 | wall |
| 138 | rack |
| 140 | rail |
| 142 | opening |
| 144 | frame |
| 146 | linear drive unit |
| 148 | housing part |
| 150 | base plate |
| 152 | fastening clips |
| 154 | projection |
| 156 | hole |
| 158 | guide rail |
| 160 | guide rail |
| 162 | guide rail |
| 164 | guide rail |
| 166 | guide track |
| 168 | drive slide |
| 170 | guide track |
| 172 | drive slide |
| 174 | guide track |
| 176 | drive slide |
| 178 | recess |
| 180 | elongate hole |
| 182 | rack |
| 184 | bearing block |
| 186 | drive motor |
| 188 | drive motor |
| 190 | drive motor |
| 192 | motor shaft |
| 194 | pinion |
| 196 | coupling device |
| 198 | rail |
| 200 | prominence |
| 202 | recess |
| 204 | opening |
| 206 | opening |
| 208 | opening |
| 210 | guide rail |
| 212 | guide rail |
| 214 | guide track |
| 216 | drive slide |
| 218 | bearing block |
| 220 | drive motor |
| 222 | opening |

What is claimed is:

1. An endoscopic instrument for the connection to an operation robot, the endoscopic instrument comprising:
an instrument housing;
a shank connected to the instrument housing at a distal side of the instrument housing;
at least one instrument part arranged on a distal side of the shank, at least one of the instrument part and the shank are movable relative to the instrument housing;
a linear drive unit connected to the instrument housing;
a translatorily movable coupling element; and
a control actively coupled to the at least one of the instrument part and the shank via the translatorily movable coupling element and to the linear drive unit.

2. An endoscopic instrument according to claim 1, wherein the coupling element is arranged in the instrument housing on a pull slide and is guided in a track-controlled manner.

3. An endoscopic instrument according to claim 1, wherein the linear drive unit comprises at least one rotatory drive motor with a rotor shaft and a connected output gear for a conversion of rotation movement of the motor shaft into translation movement.

4. An endoscopic instrument according to claim 3, wherein the gear is a rack-and-pinion gear with a rack coupled in movement to a coupling device, which coupling device is positively connectable to the coupling element on an instrument housing side.

5. An endoscopic instrument according to claim 4, wherein the rack is coupled in movement to a drive slide which is track-guided in the linear drive unit and on which the coupling device is formed.

6. An endoscopic instrument according to claim 4, wherein:
the coupling element comprises a projection which projects out of the instrument housing; and
a recess for a positive-fit receiving of the projection is formed on the coupling device.

7. An endoscopic instrument according to claim 2, wherein the coupling element on the pull slide is resiliently mounted in the connection direction of the instrument housing and the linear drive unit.

8. An endoscopic instrument according to claim 6, wherein a region, which is adjacent to the recess, forms a guide ramp for the coupling element, on a drive slide.

9. An endoscopic instrument according to claim 1, wherein at least one pull cable is provided as the control means for the instrument part, which is arranged at the distal side of the shank.

10. An endoscopic instrument according to claim 9, wherein:
the control means comprises a pull cable for the instrument part arranged on the distal side of the shank;
the pull cable has two ends connected to the instrument part in an antagonistically acting manner;
the pull cable is guided in the instrument housing in a deflection roller guide; and
a movement coupling of the pull cable to the coupling element is provided in a region between the shank and a deflection roller distanced furthest from the instrument part to be controlled, in the pull direction.

11. An endoscopic instrument according to claim 9, wherein the pull cable is connected to a pull slide which is displaceable in the pull direction of the pull cable and on which the coupling element is formed.

12. An endoscopic instrument according to claim 11, wherein a section of the pull cable, in a region of the pull slide, is formed by a pull rod.

13. An endoscopic instrument according to claim 12, wherein the pull rod, on the pull slide, is resiliently mounted on two projections which are distanced to one another in the pull direction of the pull cable.

14. An endoscopic instrument according to claim 1, further comprising a rack and a pinion, wherein the shank is coupled in movement to the pinion, which pinion in the instrument housing meshes with the rack coupled in movement to a coupling element.

15. An endoscopic instrument according to claim 12, further comprising a rack and a pinion, wherein the coupling element is formed on a pull slide which is connected to the rack.

16. An endoscopic instrument according to claim 1, wherein the instrument has movable instrument parts comprising:
an instrument head pivotably arranged on the distal end of the shank; and
on the instrument head a tool carrier which is pivotably arranged relative to the instrument head.

17. An endoscopic instrument according to claim 16, wherein the instrument as a further movable instrument part comprises a tool with two jaw parts which are pivotable relative to one another, said tool being arranged on the tool carrier.
